(19) United States
(12) Patent Application Publication
(10) Pub. No.: US 2025/0262403 A1
(43) Pub. Date: Aug. 21, 2025

(54) PORTABLE AUTOMATIC RESCUE BREATHING UNIT

(71) Applicants: Daniel A. McCarthy, Tyler, TX (US); James Crisp, Arlington, TX (US); Jorge Castillo, Liberty Hill, TX (US); Nand Mehta, Irving, TX (US); Lana Vu, Plano, TX (US); Michael Jost, The Colony, TX (US); Saad Akhtar, Irving, TX (US)

(72) Inventors: Daniel A. McCarthy, Tyler, TX (US); James Crisp, Arlington, TX (US); Jorge Castillo, Liberty Hill, TX (US); Nand Mehta, Irving, TX (US); Lana Vu, Plano, TX (US); Michael Jost, The Colony, TX (US); Saad Akhtar, Irving, TX (US)

(21) Appl. No.: 19/058,342

(22) Filed: Feb. 20, 2025

Related U.S. Application Data
(60) Provisional application No. 63/555,400, filed on Feb. 20, 2024.

(51) Int. Cl. A61M 16/20; A61M 16/00

(52) U.S. Cl. CPC A61M 16/204; A61M 16/208; A61M 2016/0039

(57) ABSTRACT

A rescue breathing apparatus and methods of operation for the rescue breathing apparatus are disclosed. The apparatus is an automatic rescue breathing unit (ARBU) device that includes a reservoir that is filled and/or drained according to alternative operations that may operate automatically or manually. One operation for filling and/or draining the reservoir is automatically controlled by a pneumatic logic circuit operating in combination with valves before and after the reservoir. A manual operation for filling the reservoir is controlled by a switch controlling flow into the reservoir.

### CROSS REFERENCE TO RELATED APPLICATIONS

This application claims priority to Provisional Patent Application Ser. No. 63/555,400 entitled “PORTABLE AUTOMATIC RESCUE BREATHING UNIT” filed Feb. 20, 2024, the entirety of which is incorporated herein by reference.

### BACKGROUND

### 1. Field of the Invention

The disclosed embodiments generally relate to a system and method for providing air/oxygen (e.g., breathing fluid) to a subject, and more particularly to a mask keying mechanism for determining breath profile and parameters to be delivered by the air/oxygen system.

### 2. Description of Related Art

Medical emergencies often call on one or more people to provide life-saving support. For example, CPR may be performed when a person is not breathing, or breathing inadequately (e.g., during cardiac arrest). CPR generally involves providing air into a person's lungs via the mouth, or mouth and nose, and/or performing a series of chest compressions. This may be performed repeatedly to help oxygenate and circulate the blood. Blowing air into the victim's mouth forces air into the lungs to replace spontaneous respiration and compressing the chest compresses the heart to maintain blood circulation. In a situation in which the heart has stopped beating, performing CPR is intended to maintain a flow of oxygenated blood to the brain and heart, thereby delaying tissue death and extending the opportunity for a successful resuscitation without permanent brain damage. Defibrillation and other advanced life support techniques may also be used to improve the outcome for a victim of cardiac arrest.

CPR techniques can vary depending on the person needing assistance. For example, administering CPR to an adult generally includes providing a set number of full breaths via the mouth, whereas administering CPR to an infant or child may require a larger number of smaller breaths or puffs via the mouth and/or nose. The lower pressure and larger numbers of breaths administered to an infant or child may reduce the likelihood of injury to the respiratory system of the infant or child. Similarly, the force used in administering the chest compressions is reduced when administering CPR to an infant or child. Accordingly, a person who administers CPR must consider several variables and remember a variety of protocols.

CPR is more effective the sooner it is initiated and thus, the time between the onset of the medical emergency and the time of initiating CPR may be critical. Brain cells may begin to die in as little as 4-6 minutes without an adequate supply of oxygen. Unfortunately, medical emergencies can, and often do, happen at locations that are remote to medical facilities and where no trained medical professionals are readily available and, thus, a by-stander may be in the best position to perform CPR.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a perspective view of an ARBU device, according to some embodiments.

FIG. 2 depicts a transparent perspective view of internal components of an ARBU device, according to some embodiments.

FIG. 3 is a block diagram overview of an ARBU device, according to some embodiments.

FIG. 4 is a flowchart illustrating a user controlled operation of an ARBU device, according to some embodiments.

FIG. 5 is a schematic depiction of the pneumatic components of an ARBU device, according to some embodiments.

FIGS. 6-9 depict operation of an ARBU device according to the schematic of FIG. 5 when a switch is deactivated, according to some embodiments.

FIG. 10 depicts schematics of the various stages of operation of an OR gate and an AND gate, according to some embodiments.

FIGS. 11 and 12 depict operation of an ARBU device according to the schematic of FIG. 5 when a switch is activated, according to some embodiments.

FIG. 13 depicts an example representation of implementation of an ARBU device in treatment of a patient, according to some embodiments.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF EMBODIMENTS

When cardiac arrest occurs, the heart stops beating. The loss of heart function causes oxygen content, the amount of usable oxygen in the blood, to be depleted. Cardiopulmonary resuscitation (CPR) on the cardiac arrest victim is required immediately. CPR consists of delivering chest compressions and air/oxygen (rescue breaths) to the patient. The goal of CPR is for the return of spontaneous circulation of blood to the body. Developments have been made in automatic rescue breathing units (ARBUs) in the treatment of patients suffering cardiac arrest to increase the chances of survival. Even with the implementation of ARBUs, however, errors can be made, even by trained professionals, that may decrease the chances of survival. Errors may, for instance, be made in the rates and volumes of rescue breaths provided to the patient due to the complexities involved in remembering and delivering appropriate rates and volumes for different types (e.g., ages or sizes) of patients. Various embodiments of ARBU devices have been described in previous patent applications. For example, example ARBU devices are found in U.S. patent application Ser. No. 18/525,346 to McCarthy et al., filed Nov. 30, 2023, which is incorporated by reference as if fully set forth herein.

The present disclosure contemplates various embodiments of an automatic rescue breathing unit (ARBU) device that includes a reservoir for building a pressure of air/oxygen for a rescue breath where filling and draining of the reservoir is automatically controlled by a pneumatic logic circuit. Draining of the reservoir provides a flow of air/oxygen (e.g., a rescue breath) to an airway device coupled to the outlet of the ARBU device. The airway device may, for instance, be placed on a face (e.g., mouth and/or nosc) of a patient to provide rescue breaths to the patient. In some embodiments, the ARBU device includes a manually controlled valve that, when activated, expedites the filling process by switching flow into the reservoir to bypass the pneumatic logic circuit when filling the reservoir. The reservoir may not be drained by the pneumatic logic circuit while the manually controlled valve is activated. When the manually controlled valve is deactivated, the pneumatic logic circuit may then operate to drain the reservoir and provide rescue breath to the patient through the outlet of the ARBU device.

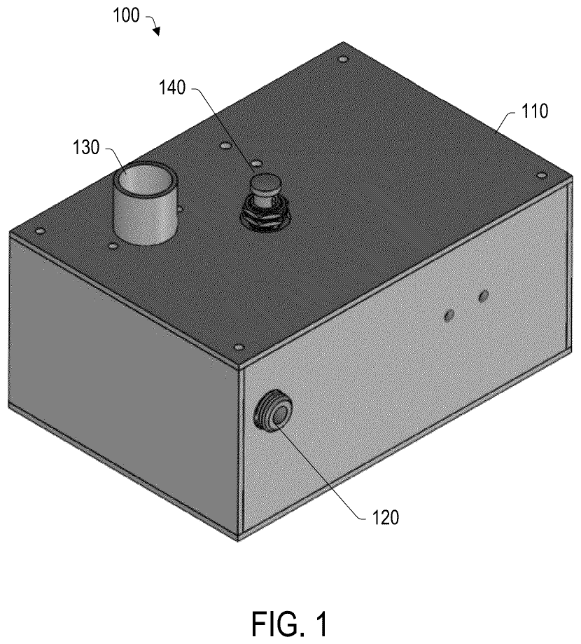

FIG. 1 depicts a perspective view of an ARBU device, according to some embodiments. In the illustrated embodiment, ARBU device 100 includes housing 110, input port 120, output port 130, and manual override switch 140. In various embodiments, as described herein, input port 120 is coupled to a source of air/oxygen (e.g., a source of breathing fluid). For instance, input port 120 may be coupled to a cylinder of pressurized oxygen or air. Output port 130 may be coupled to an airway device. As used herein, an “airway device” may include any of, but not be limited to, masks, endotracheal tubes, laryngeal mask airways, Igels, King airways, or any other airway device suitable for providing a flow or pulse of air (e.g., a rescue breath) to a patient. Manual override switch 140 may be, for example, a button or other manually operated control that can be operated to override/bypass the pneumatic logic circuit for filling a reservoir located inside ARBU device 100.

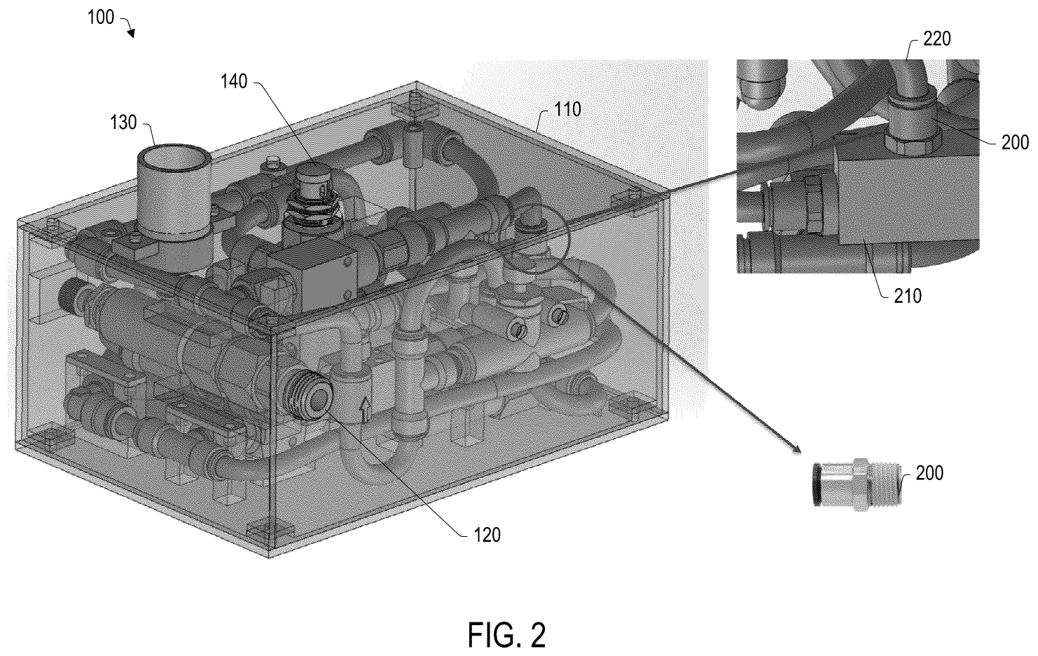

FIG. 2 depicts a transparent perspective view of internal components of an ARBU device, according to some embodiments. In the illustrated embodiment, various internal components of ARBU device as shown by example within transparent housing 110. The internal components may include, but not be limited to, connectors, manifolds, tubing, valves, gates, regulators, and tanks/reservoirs. One example component is NPT to push connector 200, shown in the insets of FIG. 2. Connector 200 may be coupled to manifold 210 to provide a connection between the manifold and tubing 220.

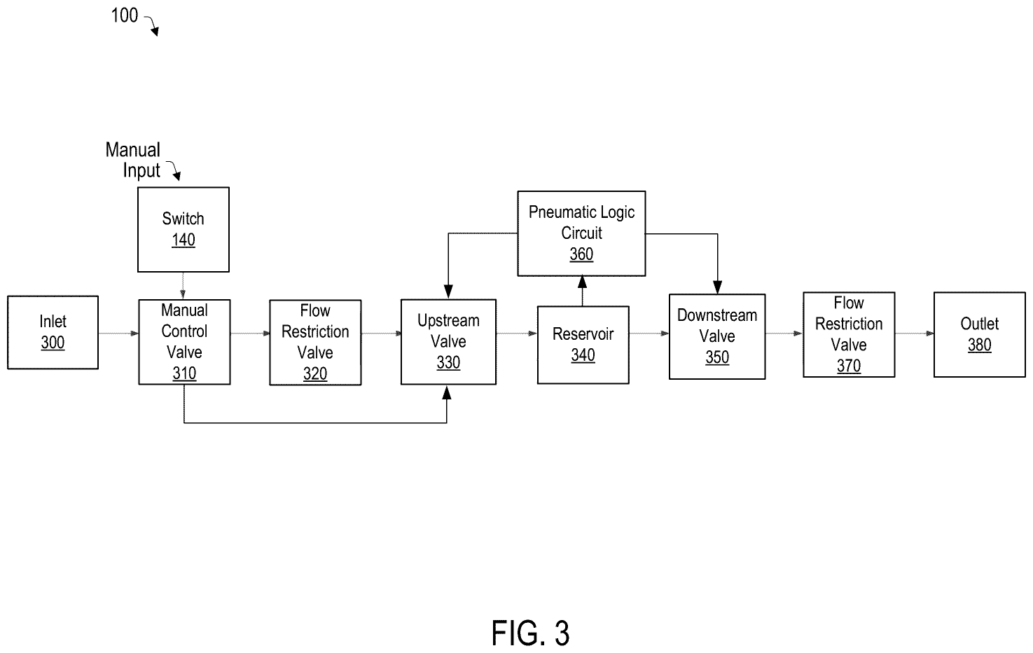

FIG. 3 is a block diagram overview of an ARBU device, according to some embodiments. In the illustrated embodiment ARBU device 100 includes inlet 300, manual control valve 310, flow restriction valve 320, upstream valve 330, reservoir 340, downstream valve 350, pneumatic logic circuit 360, flow restriction valve 370, and outlet 380. In various embodiments, a flow of air/oxygen enters ARBU device at inlet 300. The flow then passes to manual control valve 310. Manual control valve 310 may be operated according to manual input provided through switch 140. For instance, in certain embodiments, when switch 140 is not activated, manual control valve 310 is in its normal state and flow passes to flow restriction valve 320. When switch 140 is activated, manual control valve switches the flow to bypass flow restriction valve 320 and proceed directly to upstream valve 330, which, as described below, is a normally open valve thus allowing flow directly into reservoir 340.

In various embodiments, flow restriction valve 320 is a valve controls a flow of air/oxygen through the valve. For instance, flow restriction valve 320 sets a predetermined flow rate through the valve. Accordingly, the predetermined flow rate of flow restriction valve 320 may set a predetermined time period for filling reservoir 340 based on a volume (e.g., a defined volume) of the reservoir when upstream valve 330 is open and downstream valve 350 is closed. In some embodiments, the predetermined time period for filling reservoir 340 is about 5 seconds, which is determined based on the predetermined flow rate of flow restriction valve 320 and the defined volume of the reservoir.

In certain embodiments, upstream valve 330 is a normally open valve coupled to an inlet of reservoir 340. Upstream valve 330 may also be coupled to and receive control signal inputs (e.g., pneumatic control signal inputs) from pneumatic logic circuit 360. In some embodiments, a check valve may be placed between flow restriction valve 320 and upstream valve 330. As shown in FIG. 3, upstream valve 330 is coupled to the inlet of reservoir 340. Accordingly, upstream valve 330 turns on/off the flow into reservoir 340.

In various embodiments, downstream valve 350 is coupled to the outlet of reservoir 340. In certain embodiments, downstream valve 350 is a normally closed valve. Thus, downstream valve 350 normally closes off flow out of reservoir 340 and is opened to allow flow to pass through flow restriction valve 370 to outlet 380. Accordingly, operation of downstream valve 350 controls the flow of rescue breaths out of ARBU device 100 to outlet 380, which is coupled to an airway device placed on the patient.

In various embodiments, flow restriction valve 370 is a valve controls a flow of air/oxygen through the valve. For instance, flow restriction valve 370 sets a predetermined flow rate through the valve. Accordingly, the predetermined flow rate of flow restriction valve 370 may set a predetermined time period for draining reservoir 340 based on a defined volume of the reservoir when downstream valve 350 is open. In some embodiments, the predetermined time period for draining the reservoir 340 is about 1 second, which is determined based on the predetermined flow rate of flow restriction valve 370 and the defined volume of the reservoir. The time period of 1 second is an approximate time period for typical rescue breaths.

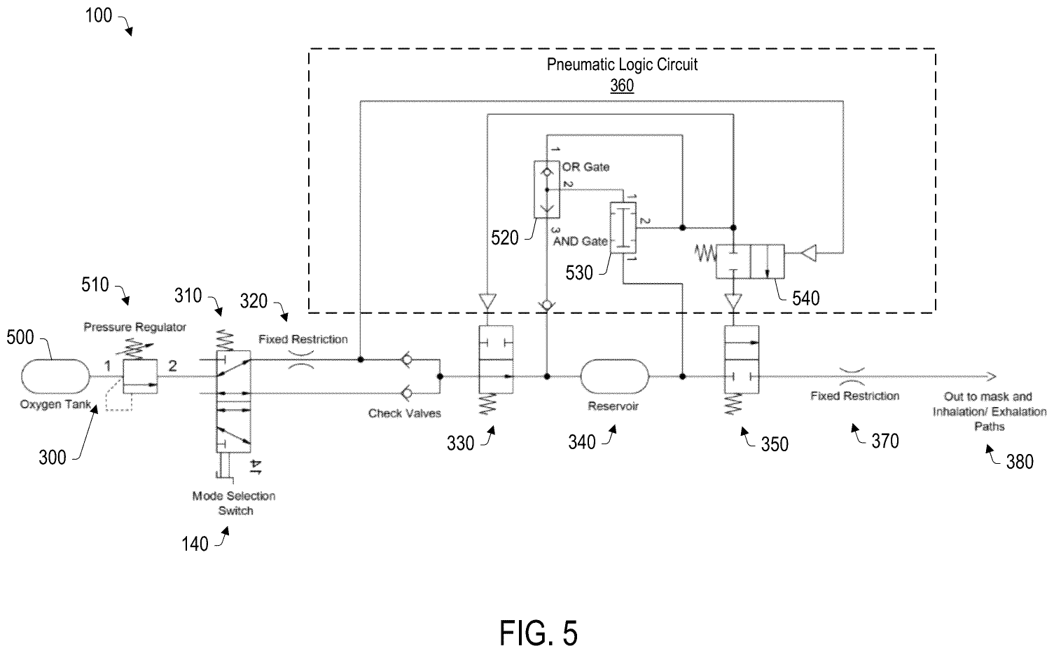

In various embodiments, as shown in FIG. 5, pneumatic logic circuit 360 is implemented to pneumatically control operation of upstream valve 330 and downstream valve 350. In certain embodiments, pneumatic logic circuit 360 includes various components that control operation of upstream valve 330 and downstream valve 350 based on pneumatic information from reservoir 340 (e.g., flow at the inlet and the outlet of the reservoir). In some embodiments, pneumatic logic circuit includes a coupling to the flow between flow restriction valve 320 and upstream valve 330 and before the check valve to pneumatically receive information about flow from restriction valve 320 to upstream valve 330.

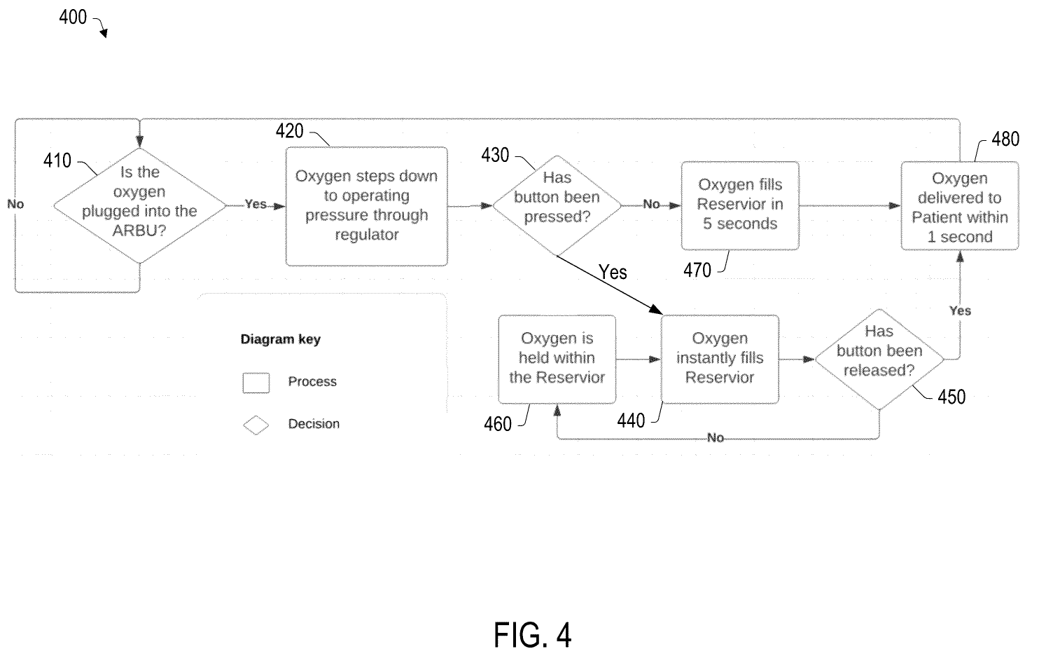

FIG. 4 is a flowchart illustrating a user controlled operation of ARBU device 100, according to some embodiments. In the illustrated embodiment, operation process 400 for ARBU device 100 begins at 410 with determining whether oxygen (or air) is plugged into (e.g., an oxygen cylinder is coupled to and opened to) the ARBU device. If “No”, then the user may wait to operate the rest of the process until oxygen is plugged in. If “Yes”, then process 400 continues at 420 with oxygen being stepped down to the proper operating pressure for ARBU device 100 by a pressure regulator (e.g., pressure regulator 510, shown in FIG. 5).

After pressure is stepped down at 420, process 400 may include whether switch 140 has been activated at 430 and manual control valve 310 is activated. As shown in FIG. 4, “Yes” at 430 sends process 400 to 440 where oxygen rapidly (e.g., “instantly”) fills reservoir 340. At 440, as described herein, activation of switch 140 and manual control valve 310 (“Yes”) sends the air/oxygen flow directly to upstream valve 330, bypassing flow restriction valve 320, to rapidly fill reservoir 340 at a fast rate (e.g., on the order of a second or less and faster than filling with flow through the flow restriction valve 320). Then at 450, it may be determined whether the button (e.g., switch 140) has been released and manual control valve is deactivated to return the flow to be through flow restriction valve 320. If “No” at 450, then oxygen is held within the reservoir at 460. Oxygen may be held within the reservoir until the button (switch 140) is released and it becomes “Yes” at 450, thus having process 400 procced to delivering oxygen to the patient by draining reservoir 340 (e.g., releasing air/oxygen from the reservoir) at 480.

Returning back to the decision at 430, when the button (e.g., switch 140) is not activated at 430 (“No”), then oxygen fills reservoir 340 according to the flow through flow restriction valve 320 (e.g., in a time period of about 5 seconds) at 470. After reservoir 340 is filled, process 400 then goes to 480 where oxygen is delivered to the patient by draining the reservoir.

FIG. 5 is a schematic depiction of the pneumatic components of ARBU device 100, according to some embodiments. In the illustrated embodiment, oxygen tank 500 is coupled to ARBU device 100. ARBU device 100 includes inlet 300, pressure regulator 510, manual control valve 310, flow restriction valve 320, upstream valve 330, reservoir 340, downstream valve 350, pneumatic logic circuit 360, flow restriction valve 370, and outlet 380.

In certain embodiments, pneumatic logic circuit 360 includes pneumatic OR gate 520, pneumatic AND gate 530, and normally closed valve 540. As shown in FIG. 5, one of the inputs of OR gate 520 (“3”) is coupled to the inlet of reservoir 340. The other input of OR gate 520 (“1”) is coupled to the output of AND gate 530 (“2”). The output of OR gate 520 (“2”) is coupled to one of the inputs of AND gate 530 (“1”). The output of AND gate 530 (“2”) is also coupled to a control input of upstream valve 330 and the inlet of normally closed valve 540. The other input of AND gate 530 (“1”) is coupled to the outlet of reservoir 340. The control input of normally closed valve 540 is coupled to the flow between flow restriction valve 320 and upstream valve 330. The outlet of normally closed valve 540 is coupled to the control input of downstream valve 350.

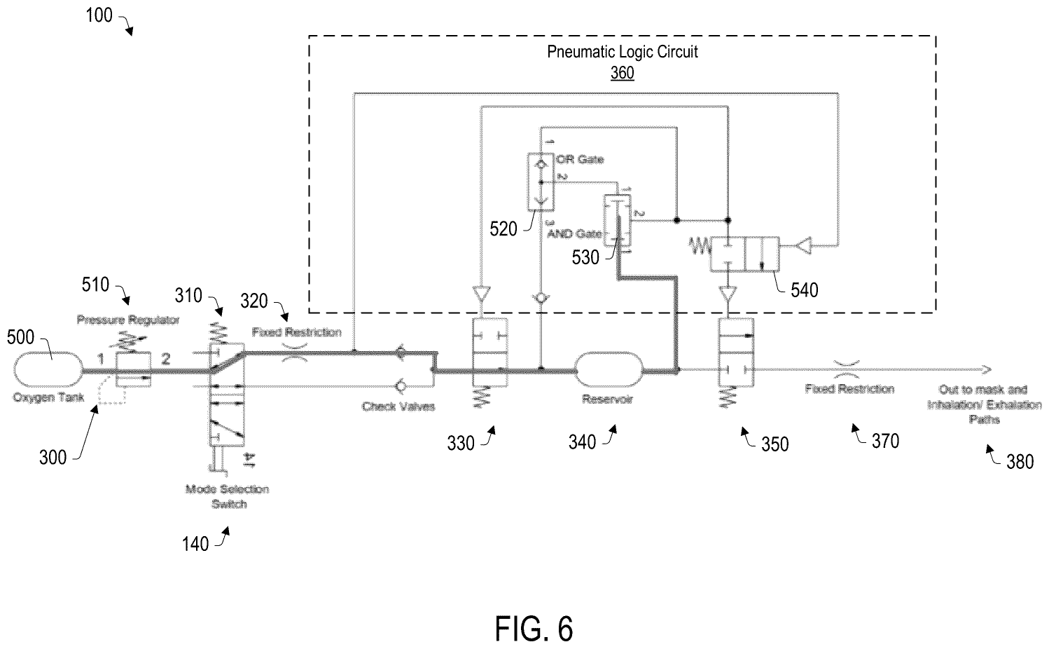

FIGS. 6-9 depict operation of ARBU device 100 according to the schematic of FIG. 5 when switch 140 is deactivated (e.g., manual control valve 310 is not activated), according to some embodiments. Flow of oxygen (or air) is shown by the thickened lines in the illustrated embodiments. As shown in FIG. 6-9, with switch and manual control valve 310 not activated, flow of oxygen goes through flow restriction valve 320. For instance, as shown in FIG. 6, flow initially proceeds from oxygen tank 500, through pressure regulator 510, through manual control valve 310, through flow restriction valve 320, and through upstream valve 330 (normally open) to reservoir 340. With downstream valve 350 being normally closed, a pneumatic signal from the outlet of reservoir 340 is received at the input to AND gate 530.

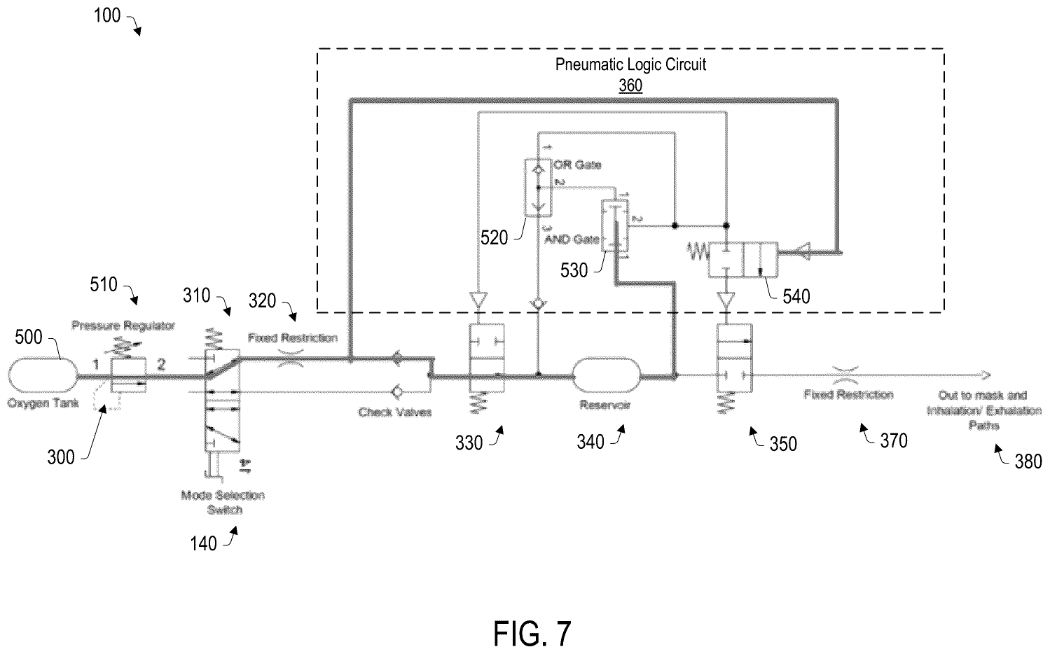

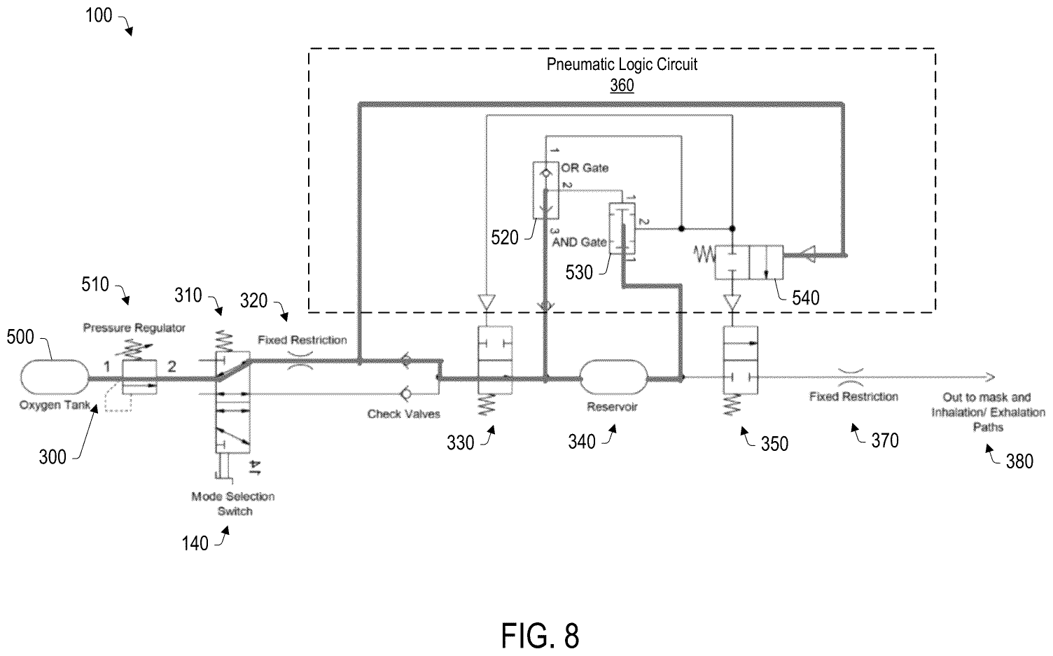

Flow continues into reservoir 340 until the reservoir is filled. When reservoir 340 is filled, the excess flow provides a pneumatic signal to the control input of normally closed valve 540 in pneumatic logic circuit 360 from the connection between flow restriction valve 320 and upstream valve 330, as shown in FIG. 7. The control input at normally closed valve 540 causes the valve to open. Soon after, or around the same time, the excess flow also provides a pneumatic signal from the inlet of reservoir 340 to the input of OR gate 520, as shown in FIG. 8. This provides an indication that reservoir 340 is at full pressure (e.g., the reservoir is fully filled to a maximum pressure and/or the reservoir is filled to where the breathing fluid (air/oxygen) stops flowing into the reservoir).

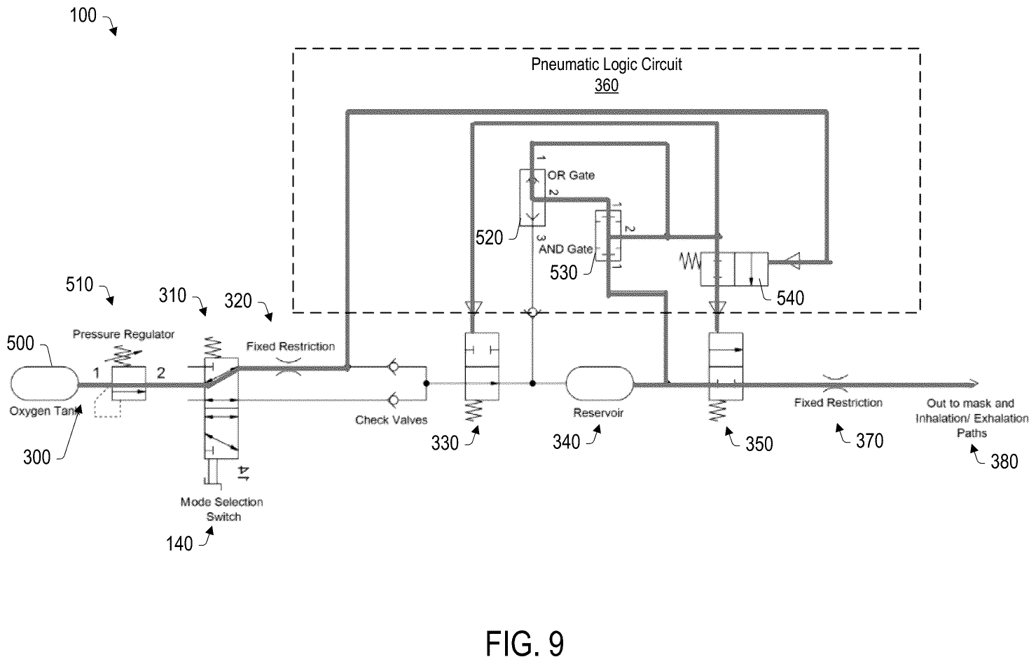

With the pneumatic signal input received at OR gate 520, OR gate 520 then provides an output pneumatic signal to AND gate 530, as shown in FIG. 9. As AND gate 530 is already receiving pneumatic signal input from the outlet of reservoir 340 at its other input, AND gate 530 then outputs a pneumatic signal. The pneumatic signal output of AND gate 530 is received at the other input of OR gate 520, which continues the pneumatic signal output of the OR gate. The pneumatic signal of AND gate 530 is also sent to the control input of upstream valve 330, which causes the upstream valve to close. The pneumatic signal output of AND gate 530 is further sent to the inlet of normally closed valve 540, which is open based on receiving the pneumatic signal from the flow between flow restriction valve and upstream valve 330. Accordingly, the pneumatic signal of AND gate 530 passes through normally closed valve 540 and is received at the control input of downstream valve 350, which causes the downstream valve to open and send flow of oxygen (or air) from reservoir 340 through flow restriction valve 370 and to outlet 380 until the reservoir is drained. After reservoir 340 is drained of air/oxygen (e.g., the breathing fluid (air/oxygen) stops flowing out of the reservoir), AND gate 530 will reset, which causes resetting of all the gates and valves back to the original state to wait for the initial flow of air/oxygen, as shown in FIG. 6. In some embodiments, reservoir 340 is considered to be “drained of air/oxygen” when the a pressure in the reservoir is below a minimum pressure for flow (e.g., a pressure below which flow of air/oxygen out of the reservoir is stopped).

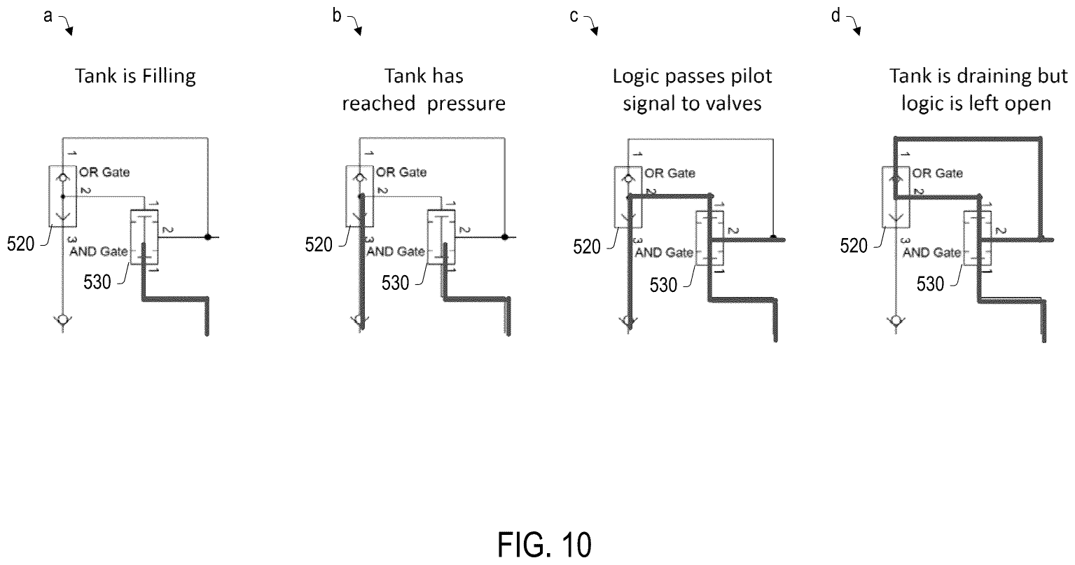

FIG. 10 depicts schematics of the various stages of operation of OR gate 520 and AND gate 530, according to some embodiments. In (a), reservoir 340 is filling and a pneumatic signal is only received at one of the inputs to AND gate 530. In (b), reservoir 340 has reached full pressure and pneumatic signals are received at the inputs to both OR gate 520 and AND gate 530. At (c), the pneumatic signal output of OR gate 520 is received at AND gate 530 such that the AND gate provides an output pneumatic signal to coupled valves and the other input of the OR gate. At (d), with the pneumatic signal output of AND gate 530 being received at one of the inputs of OR gate 520, the pneumatic logic is left open while reservoir 340 drains. As described above, the pneumatic logic remains open until reservoir 340 fully drains, at which point the pneumatic signal at the lower input of AND gate 530 is turned off and the pneumatic logic closes and resets the system.

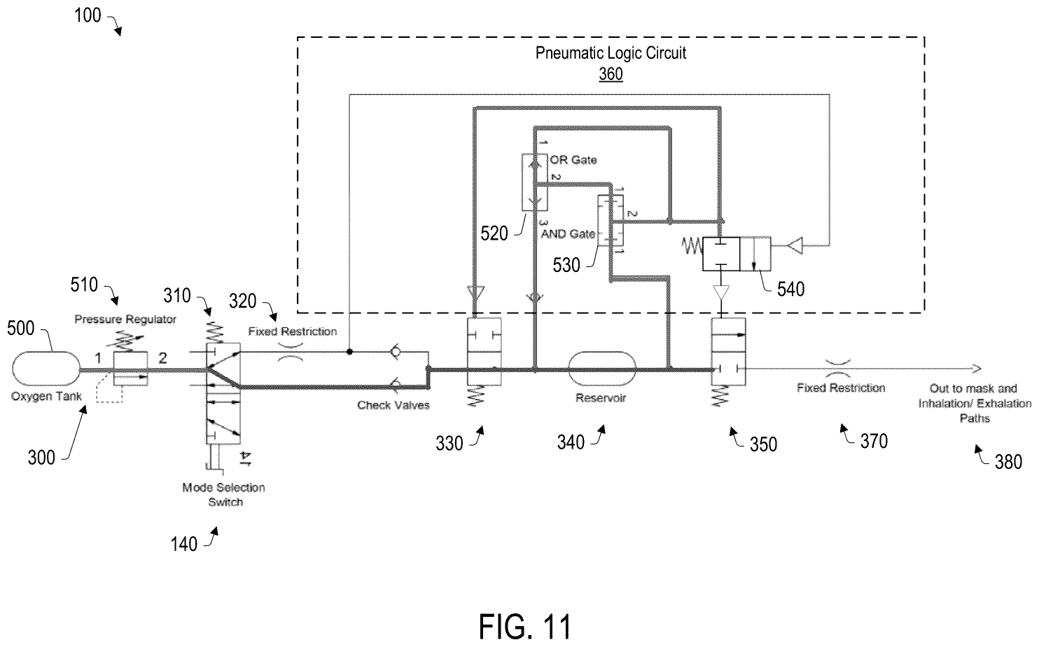

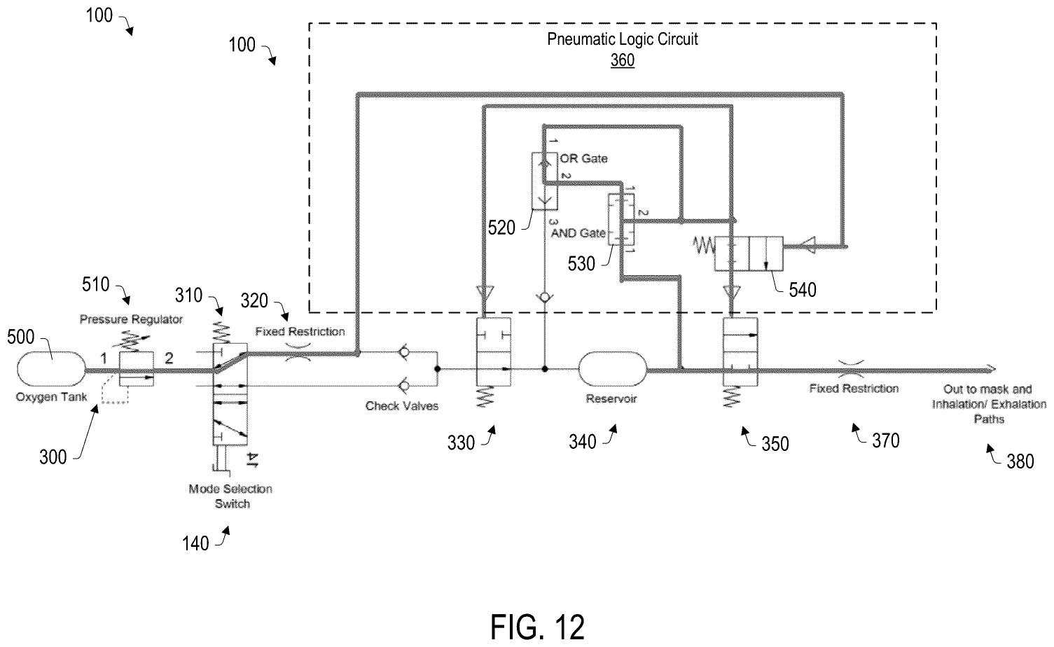

FIGS. 11 and 12 depict operation of ARBU device 100 according to the schematic of FIG. 5 when switch 140 is activated (e.g., manual control valve 310 is activated), according to some embodiments. Flow of oxygen (or air) is shown by the thickened lines in the illustrated embodiments. As shown in FIG. 11, with switch 140 and manual control valve 310 activated, flow of oxygen a second flow line connected to upstream valve 330 that bypasses flow restriction valve 320. With the bypassing of flow restriction valve 320, reservoir 340 fills rapidly (e.g., on the order of a second or less) and pneumatic signal inputs are quickly received at the inputs of OR gate 520 and AND gate 530 from the inlet and outlet of reservoir 340, respectively. Accordingly, a pneumatic signal is received at upstream valve 330 to close the valve. However, normally closed valve 540 remains closed because there is no pneumatic signal input at the control input to normally closed valve 540 and thus the pneumatic signal from the output of AND gate 530 is not passed through the valve to downstream valve 350.

Normally closed valve 540 remains closed as long as switch 140 and manual control valve 310 are activated since there is no connection to the flow line bypassing flow restriction valve 320. Thus, in order for a breath to be released (e.g., downstream valve 350 to be opened), switch 140 and manual control valve 310 need to be deactivated (e.g., the button of switch 140 is released). When switch 140 and manual control valve 310 are deactivated, flow is returned to the flow line of flow restriction valve 320 and the pneumatic signal is received at normally closed valve 540, opening the valve and providing the pneumatic signal from the output of AND gate 530 to the control input of downstream valve 350, as shown in FIG. 12. Note that the schematic of FIG. 12 and the providing of the rescue breath is substantially similar to the schematic and breath providing depicted in FIG. 9.

As described herein, ARBU device 100 is a device capable of providing controlled volumes of breath to a patient in a small form factor. ARBU device 100 includes a reservoir with a defined volume that is filled to provide a specified volume of air/oxygen to the patient at a predetermined flow rate. Additionally, ARBU device 100 allows for a user to manually control the device to provide rapid filling of the reservoir and then release the breath to the patient based on the manual control.

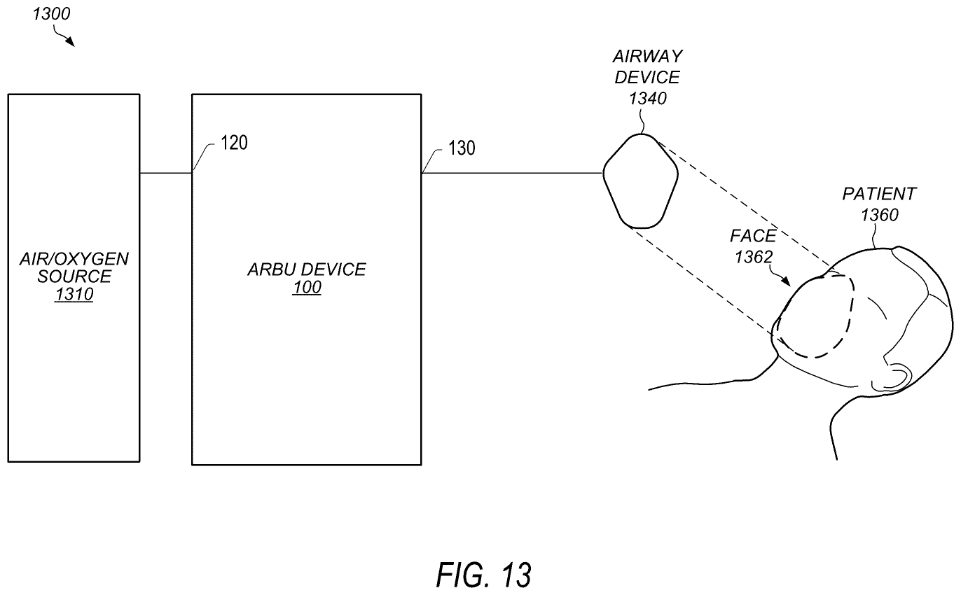

FIG. 13 depicts an example representation of implementation of ARBU device in treatment of patient 1360, according to some embodiments. In the illustrated embodiment of ARBU system 1300, device 100 is coupled to air/oxygen source 1310. Device 100 is being implemented in the treatment of patient 1360, shown with face 1362. In various embodiments, device 100 is a portable system that is transported to a location of patient 1360.

In various embodiments, source 1310 is coupled to input port 120 (e.g., inlet) of device 100. Source 1310 is a source capable of providing pressurized air/oxygen for use by device 100. As used herein “pressurized air/oxygen” refers to air/oxygen having a pressure that promotes the flow of the air/oxygen from device 100 into the lungs of patient 1360. In some embodiments, pressurized air/oxygen may have a pressure that is above a minimum-pressure threshold, such as 20 centimeters of water (cmH₂O) above ambient air pressure.

In certain embodiments, source 1310 is a cylinder containing pressurized air/oxygen. The pressure of the air/oxygen may be set significantly above the minimum-pressure threshold such that the air/oxygen in the cylinder is maintained above the minimum-pressure threshold as the air/oxygen is expelled from the cylinder and the pressure of the air/oxygen in the source 1310 drops as a function of the air/oxygen expelled from the cylinder. Source 1310 may include a mechanical device, such as a compressor, configured to move and/or pressurize the air/oxygen. Such a mechanical device may be used to pressurize and/or fill a cylinder of the source 1310. In one embodiment, source 1310 may include the mechanical device to move the air from the cylinder to the subject.

During treatment (e.g., resuscitation) of patient 1360, airway device 1340 (e.g., a mask) is coupled to face 1362, or applied to the airway, of the patient. Airway device 1340 is coupled to output port 130 (e.g., outlet) of ARBU device 100. In various embodiments, ARBU device 100 provides one or more rescue breaths to patient 1360 through airway device 1340 according to the embodiments described herein.

The order of the methods may be changed, and various elements may be added, reordered, combined, omitted, modified, etc. Various modifications and changes may be made as would be obvious to a person skilled in the art having the benefit of this disclosure. The various embodiments described herein are meant to be illustrative and not limiting. Many variations, modifications, additions, and improvements are possible. Accordingly, plural instances may be provided for components described herein as a single instance. Boundaries between various components, operations and data stores are somewhat arbitrary, and particular operations are illustrated in the context of specific illustrative configurations. Other allocations of functionality are envisioned and may fall within the scope of claims that follow. Finally, structures and functionality presented as discrete components in the example configurations may be implemented as a combined structure or component. These and other variations, modifications, additions, and improvements may fall within the scope of embodiments as defined in the claims that follow.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. The words “include”, “including”, and “includes” mean including, but not limited to. As used throughout this application, the singular forms “a”, “an” and “the” include plural referents unless the content clearly indicates otherwise. Thus, for example, reference to “a mask” includes a combination of two or more masks. The term “coupled” means directly or indirectly connected.

What is claimed is:

1. A rescue breath apparatus, comprising:
a ventilator device configured to be coupled to a breathing fluid source, wherein the ventilator device includes:
a fluid inlet configured to be coupled to the breathing fluid source;
a pressure regulator configured to control a pressure of the breathing fluid entering the apparatus through the fluid inlet;
a reservoir having a defined volume, the reservoir having an inlet and an outlet;
an upstream valve coupled to the inlet of the reservoir;
a downstream valve coupled to the outlet of the reservoir;
a pneumatic flow logic circuit coupled to the reservoir, the upstream valve, and the downstream valve, wherein the pneumatic flow logic circuit is configured to control operation of the upstream valve and the downstream valve to fill the reservoir with the breathing fluid and drain the reservoir of the breathing fluid based on a flow of the breathing fluid into and out of the reservoir;
a manually controlled valve coupled between the pressure regulator and the upstream valve, wherein the manually controlled valve, when activated, bypasses the pneumatic flow logic circuit to fill the reservoir; and
a fluid outlet coupled to the downstream valve, wherein the outlet is configured to be coupled to an airway device for a patient.

2. The apparatus of claim 1, further comprising:
a first flow control valve coupled between the pressure regulator and the upstream valve; and
a second flow control valve coupled between the downstream valve and the outlet.

3. The apparatus of claim 2, wherein the manually controlled valve bypasses the first flow control valve to fill the reservoir when activated.

4. The apparatus of claim 2, wherein the first flow control valve restricts flow from the pressure regulator to the upstream valve to set a predetermined time period for filling the reservoir through the upstream valve.

5. The apparatus of claim 4, wherein the predetermined time period for filling the reservoir is about 5 seconds.

6. The apparatus of claim 2, wherein the second flow control valve restricts flow from the downstream valve to the outlet to set a predetermined time period for draining the reservoir through the downstream valve.

7. The apparatus of claim 6, wherein the predetermined time period for draining the reservoir is about 1 second.

8. The apparatus of claim 1, wherein the upstream valve is a normally open valve and the downstream valve is a normally closed valve.

9. The apparatus of claim 8, wherein the pneumatic flow logic circuit includes:
a pneumatic OR gate having:
a first input coupled between the upstream valve and the inlet of the reservoir;
a second input; and
an output;
a pneumatic AND gate having:
a first input coupled between the outlet of the reservoir and the downstream valve;
a second input coupled to the output of the pneumatic OR gate; and
an output coupled to the second input of the pneumatic OR gate and a control input of the upstream valve;
a second normally closed valve having:
an inlet coupled to the manually controlled valve and configured to receive flow then the manually controlled valve is not activated;
a control input coupled to the output of pneumatic AND gate; and
an outlet coupled to a control input of the downstream valve.

10. The apparatus of claim 9, wherein the control input of the upstream valve closes the upstream valve in response to receiving a pneumatic signal from the output of the pneumatic AND gate.

11. The apparatus of claim 9, wherein the control input of the downstream valve opens the downstream valve in response to receiving a pneumatic signal from the outlet of the second normally closed valve.

12. The apparatus of claim 1, wherein the manually controlled valve switches flow between a first flow line, when not activated, and a second flow line, when activated, according to a manual actuation of the valve.

13. The apparatus of claim 12, wherein the first flow line includes a first flow control valve coupled between the pressure regulator and the upstream valve, and wherein the first flow line is coupled to a second normally closed valve in the pneumatic flow logic circuit and to the upstream valve.

14. The apparatus of claim 12, wherein the second flow line is directly coupled to the upstream valve.

15. The apparatus of claim 12, further comprising check valves in the first flow line and the second flow line.

16. The apparatus of claim 1, wherein the manually controlled valve inhibits opening of the downstream valve and draining of the reservoir when activated.

17. The apparatus of claim 1, wherein the pneumatic flow logic circuit is configured to control operation of the upstream valve and the downstream valve to fill the reservoir with the breathing fluid and drain the reservoir of the breathing fluid based on a flow of the breathing fluid into and out of the reservoir in combination with the flow of the breathing fluid through the upstream valve and the downstream valve.

18. The apparatus of claim 1, wherein the pneumatic flow logic circuit is configured to fill the reservoir with the breathing fluid until the reservoir is filled until the breathing fluid stops flowing into the reservoir and drain the reservoir of the breathing fluid until the breathing fluid stops flowing out of the reservoir.

19. A rescue breath apparatus, comprising:
a ventilator device configured to be coupled to a breathing fluid source, wherein the ventilator device includes:
a fluid inlet configured to be coupled to the breathing fluid source;
a pressure regulator configured to control a pressure of the breathing fluid entering the apparatus through the fluid inlet;
a reservoir having a defined volume, the reservoir having an inlet and an outlet;
an upstream valve coupled to the inlet of the reservoir;
a downstream valve coupled to the outlet of the reservoir;
a pneumatic flow logic circuit coupled to the reservoir, the upstream valve, and the downstream valve, wherein the pneumatic flow logic circuit is configured to control opening and closing of both the upstream valve and the downstream valve to fill the reservoir with the breathing fluid and drain the reservoir of the breathing fluid based on a flow of the breathing fluid through a combination of the pneumatic flow logic circuit, the upstream valve, and the downstream valve;
a manually controlled valve coupled between the pressure regulator and the upstream valve, wherein the manually controlled valve, when activated, bypasses the pneumatic flow logic circuit to fill the reservoir; and
a fluid outlet coupled to the downstream valve, wherein the outlet is configured to be coupled to an airway device for a patient.

20. The apparatus of claim 19, wherein the pneumatic flow logic circuit is configured to fill the reservoir with the breathing fluid until the reservoir is at full pressure and drain the reservoir of the breathing fluid until a pressure of the reservoir is below a minimum pressure for flow.
